(19) United States
(12) Patent Application Publication
(10) Pub. No.: US 2004/0248779 A1
(43) Pub. Date: Dec. 9, 2004

(54) Process for the preparation of macrocyclic compounds

(75) Inventors: Wolfgang Dersch, Ingelheim (DE); Wendelin Samstag, Bad Kreuznach (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim International GmbH, Ingelheim (DE)

(21) Appl. No.: 10/813,344

(22) Filed: Mar. 30, 2004

Related U.S. Application Data
(60) Provisional application No. 60/461,879, filed on Apr. 10, 2003.

(51) Int. Cl. A61K 38/12; C07K 5/12

(52) U.S. Cl. 514/9; 514/312; 530/317

(57) ABSTRACT

The invention relates to an improved process for the preparation of a macrocyclic compound of formula I

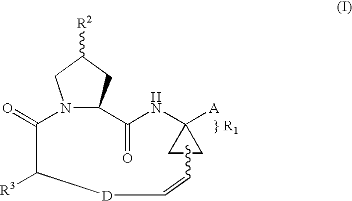

wherein R₁, R², R³, A and D have the meaning given in the claims; by a ring closure metathesis of the corresponding diene of formula III

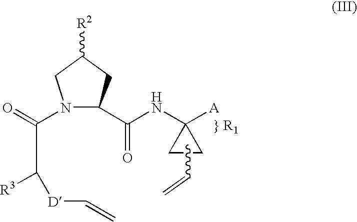

wherein R₁, R², R³, A and D′ have the meaning given in the claims; in the presence of a benzylidene ruthenium catalyst, wherein the phenyl group is substituted by a nitro group.

This application claims benefit from U.S. Provisional Application No. 60/461,879, filed Apr. 10, 2003, which application is herein incorporated by reference in its entirety.

### BACKGROUND OF THE INVENTION

1. Technical Field

The invention relates to an improved process for the preparation of macrocyclic compounds of formula I

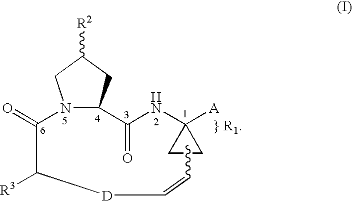

2. Background Information

The macrocyclic compounds of formula I are known from the International patent application WO 00/59929. The compounds disclosed there are highly active agents for the treatment of hepatitis C virus infections. The methods for the preparation of these compounds include many synthetic steps, which involve protection and deprotection of certain reactive groups and leads to an insufficient overall yield. Moreover, the International patent application suggests to form the macrocycle via an olefin metathesis using a ruthenium based catalyst, selected from the following formulae

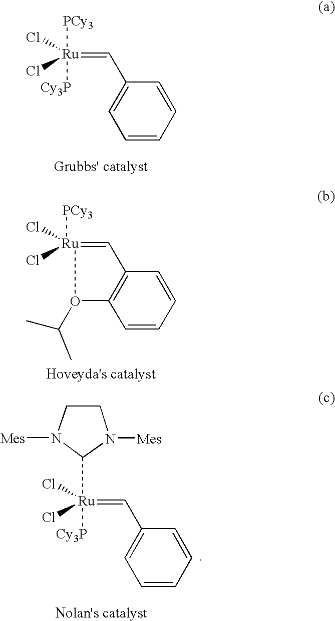

Unfortunately, this reaction can only be carried out in extremely diluted reaction systems and takes a very long time for completion. Moreover, comparably high amounts of these catalysts (5.5 to 30 mol %) are necessary to complete the reaction.

Recently, K. Grela et al., Angew. Chem. Int. Ed. 2002, 41, No. 21 pp. 4038-4040 have suggested a new benzylidene ruthenium catalyst in which the phenyl group is substituted by a nitro group.

The problem underlying the present invention was to provide a process which allows the manufacture of the macrocyclic compounds of formula I in a technical scale with lower amounts of catalyst, better turn-over rates, higher yields and improved room-time yield.

Surprisingly it has been found that a better turn-over rate with less undesired by-products can be achieved when the cyclisation metathesis reaction is carried out with a benzylidene ruthenium catalyst, in which the phenyl group of the benzylidene group is substituted by a nitro group, which can efficiently be used in an amount of less than 1 mol %.

### BRIEF SUMMARY OF THE INVENTION

Therefore, the invention relates to an improved process for the preparation of a macrocyclic compound of formula I

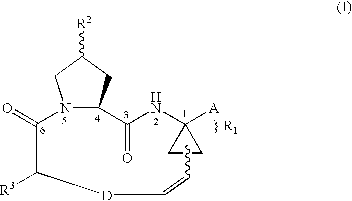

wherein

R² is a hydroxy group, a leaving group or a group of formula II

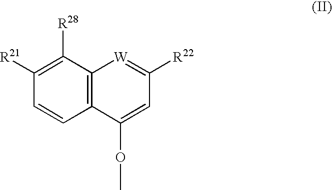

W is CH or N,

R²¹ is H, halo, C₁₋₆ alkyl, C₃₋₆ cycloalkyl, C₁₋₆ haloalkyl, C₁₋₆ alkoxy, C₃₋₆ cycloalkoxy, hydroxy, or N(R²³)₂,

wherein each R²³ is independently H, C₁₋₆ alkyl or C₃₋₆ cycloalkyl;

R²² is H, halo, C₁₋₆ alkyl, C₃₋₆ cycloalkyl, C₁₋₆ haloalkyl, C₁₋₆ thioalkyl, C₁₋₆ alkoxy, C₃₋₆ cycloalkoxy, C₂₋₇ alkoxyalkyl, C₃₋₆ cycloalkyl, C_{6 or 10} aryl or Het, wherein Het is a five-, six-, or seven-membered saturated or unsaturated heterocycle containing from one to four heteroatoms selected from nitrogen, oxygen and sulfur;

said cycloalkyl, aryl or Het being substituted with R²⁴, wherein

R²⁴ is H, halo, C₁₋₆ alkyl, C₃₋₆ cycloalkyl, C₁₋₆ alkoxy, C₃₋₆ cycloalkoxy, NO₂, N(R²⁵)₂, NH—C(O)—R²⁵; or NH—C(O)—NH—R²⁵, wherein each R²⁵ is independently: H, C₁₋₆ alkyl or C₃₋₆ cycloalkyl; or

R²⁴ is NH—C(O)—OR²⁶ wherein R²⁶ is C₁₋₆ alkyl or C₃₋₆ cycloalkyl;

R²⁸ is H, halo or C₁₋₆ alkyl, preferably H

R³ is hydroxy, NH₂, or a group of formula —NH—R³¹, wherein R³¹ is C_{6 or 10} aryl, heteroaryl, —C(O)—R³², —C(O)—NHR³² or —C(O)—OR³², wherein R³² is C₁₋₆ alkyl or C₃₋₆ cycloalkyl;

D is a 3 to 7-atom saturated alkylene chain; and

A is an amide of formula —C(O)—NH—R⁵, wherein R⁵ is selected from the group consisting of: C₁₋₈ alkyl, C₃₋₆ cycloalkyl, C_{6 or 10} aryl, C₇₋₁₆ aralkyl; and SO₂R^{5A} wherein R^{5A} is C₁₋₈ alkyl, C₃₋₇ cycloalkyl or {C₁₋₆ alkyl-C₃₋₇ cycloalkyl}, or

A is a carboxylic acid or a pharmaceutically acceptable salt or ester thereof;

which process comprises subjecting a diene compound of formula III

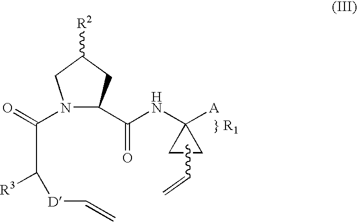

wherein R², R³ and A are as defined hereinbefore; and

D′ represents a 3 to 7-atom saturated alkylene chain;

to a metathesis cyclization reaction in the presence of a ruthenium catalyst of formula IV,

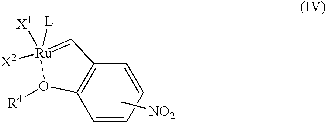

wherein

X¹ and X² each independently represent an anionic ligand;

L represents a neutral electron donor ligand; and

R⁴ represents a C₁₋₆ alkyl, C₂₋₆ alkenyl or C₆₋₁₂ aryl-C₁₋₆ alkyl group.

### DETAILED DESCRIPTION OF THE INVENTION

### Definition of Terms and Conventions Used

Terms not specifically defined herein should be given the meanings that would be given to them by one of skill in the art in light of the disclosure and the context. As used in the specification, however, unless specified to the contrary, the following terms have the meaning indicated and the following conventions are adhered to.

In the groups, radicals, or moieties defined below, the number of carbon atoms is often specified preceding the group, for example, C₁₋₆ alkyl means an alkyl group or radical having 1 to 6 carbon atoms. In general, for groups comprising two or more subgroups, the last named group is the radical attachment point, for example, “thioalkyl” means a monovalent radical of the formula HS-Alk-. Unless otherwise specified below, conventional definitions of terms control and conventional stable atom valences are presumed and achieved in all formulas and groups.

The term “C₁₋₆ alkyl” as used herein, either alone or in combination with another substituent, means acyclic, straight or branched chain alkyl substituents containing from 1 to six carbon atoms and includes, for example, methyl, ethyl, propyl, butyl, hexyl, 1-methylethyl, 1-methylpropyl, 2-methylpropyl, and 1,1-dimethylethyl.

The term “C₃₋₆ cycloalkyl” as used herein, either alone or in combination with another substituent, means a cycloalkyl substituent containing from three to six carbon atoms and includes cyclopropyl, cyclobutyl, cyclopentyl, and cyclohexyl.

The term “saturated alkylene chain” as used herein means a divalent alkyl substituent derived by the removal of one hydrogen atom from each end of a saturated straight or branched chain aliphatic hydrocarbon and includes, for example, CH₂CH₂C(CH₃)₂CH₂CH₂—.

The term “C₁₋₆ alkoxy” as used herein, either alone or in combination with another substituent, means the substituent C₁₋₆ alkyl-O— wherein alkyl is as defined above containing up to six carbon atoms. Alkoxy includes methoxy, ethoxy, propoxy, 1-methylethoxy, butoxy and 1,1-dimethylethoxy. The latter substituent is known commonly as tert-butoxy.

The term “C₃₋₆ cycloalkoxy” as used herein, either alone or in combination with another substituent, means the substituent C₃₋₆ cycloalkyl-O— containing from 3 to 6 carbon atoms.

The term “C₂₋₇ alkoxy-C₁₋₆ alkyl” as used herein, means the substituent C₂₋₇ alkyl-O—C₁₋₆ alkyl wherein alkyl is as defined above containing up to six carbon atoms.

The term “halo” as used herein means a halogen substituent selected from bromo, chloro, fluoro or iodo.

The term “haloalkyl” as used herein means as used herein, either alone or in combination with another substituent, means acyclic, straight or branched chain alkyl substituents having one or more hydrogens substituted for a halogen selected from bromo, chloro, fluoro or iodo.

The term “thioalkyl” as used herein means as used herein, either alone or in combination with another substituent, means acyclic, straight or branched chain alkyl substituents containing a thiol (HS) group as a substituent. An example of a thioalkyl group is a thiopropyl, e.g., HS—CH₂CH₂CH₂— is one example of a thiopropyl group.

The term “C₆ or C₁₀ aryl” as used herein, either alone or in combination with another substituent, means either an aromatic monocyclic system containing 6 carbon atoms or an aromatic bicyclic system containing 10 carbon atoms. For example, aryl includes a phenyl or a naphthyl—ring system.

The term “C₇₋₁₆ aralkyl” as used herein, either alone or in combination with another substituent, means an aryl as defined above linked through an alkyl group, wherein alkyl is as defined above containing from 1 to 6 carbon atoms. Aralkyl includes for example benzyl, and butylphenyl.

The term “Het” as used herein, either alone or in combination with another substituent, means a monovalent substituent derived by removal of a hydrogen from a five-, six-, or seven-membered saturated or unsaturated (including aromatic) heterocycle containing carbon atoms and from one to four ring heteroatoms selected from nitrogen, oxygen and sulfur. Examples of suitable heterocycles include: tetrahydrofuran, thiophene, diazepine, isoxazole, piperidine, dioxane, morpholine, pyrimidine or

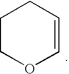

The term “Het” also includes a heterocycle as defined above fused to one or more other cycle be it a heterocycle or any other cycle. One such examples includes thiazolo[4,5-b]-pyridine. Although generally covered under the term “Het”, the term “heteroaryl” as used herein precisely defines an unsaturated heterocycle for which the double bonds form an aromatic system. Suitable example of heteroaromatic system include: quinoline, indole, pyridine,

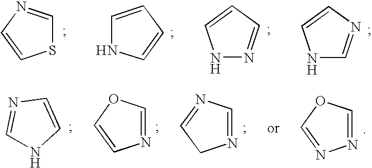

The term “oxo” means the double-bonded group (═O) attached as a substituent.

The term “thio” means the double-bonded group (═S) attached as a substituent.

In general, all tautomeric forms and isomeric forms and mixtures, whether individual geometric isomers or optical isomers or racemic or non-racemic mixtures of isomers, of a chemical structure or compound is intended, unless the specific stereochemistry or isomeric form is specifically indicated in the compound name or structure.

The term “pharmaceutically acceptable ester” as used herein, either alone or in combination with another substituent, means esters of the compound of formula I in which any of the carboxyl functions of the molecule, but preferably the carboxy terminus, is replaced by an alkoxycarbonyl function:

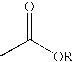

in which the R moiety of the ester is selected from alkyl (e.g. methyl, ethyl, n-propyl, t-butyl, n-butyl); alkoxyalkyl (e.g. methoxymethyl); alkoxyacyl (e.g. acetoxymethyl); aralkyl (e.g. benzyl); aryloxyalkyl (e.g. phenoxymethyl); aryl (e.g. phenyl), optionally substituted with halogen, C₁₋₄ alkyl or C₁₋₄ alkoxy. Other suitable prodrug esters are found in Design of prodrugs, Bundgaard, H. Ed. Elsevier (1985) incorporated herewith by reference. Such pharmaceutically acceptable esters are usually hydrolyzed in vivo when injected in a mammal and transformed into the acid form of the compound of formula I. With regard to the esters described above, unless otherwise specified, any alkyl moiety present advantageously contains 1 to 16 carbon atoms, particularly 1 to 6 carbon atoms. Any aryl moiety present in such esters advantageously comprises a phenyl group. In particular the esters may be C₁₋₁₆ alkyl ester, an unsubstituted benzyl ester or a benzyl ester substituted with at least one halogen, C₁₋₆ alkyl, C₁₋₆ alkoxy, nitro or trifluoromethyl. The term “pharmaceutically acceptable salt” as used herein includes those derived from pharmaceutically acceptable bases. Examples of suitable bases include choline, ethanolamine and ethylenediamine. Na⁺, K⁺, and Ca⁺⁺ salts are also contemplated to be within the scope of the invention (also see Pharmaceutical salts, Birge, S. M. et al., J. Pharm. Sci., (1977), 66, 1-19, incorporated herein by reference).

### EMBODIMENTS OF THE INVENTION

In the synthetic schemes below, unless specified otherwise, all the substituent groups in the chemical formulas shall have the same meanings as in the Formula (I). The reactants used in the synthetic schemes described below may be obtained either as described herein, or if not described herein, are themselves either commercially available or may be prepared from commercially available materials by methods known in the art. Certain starting materials, for example, may be obtained by methods described in the International Patent Applications WO 00/59929, WO 00/09543 and WO 00/09558, U.S. Pat. No. 6,323,180 B1 and U.S. Pat. No. 6,608,027 B1.

Optimum reaction conditions and reaction times may vary depending on the particular reactants used. Unless otherwise specified, solvents, temperatures, pressures, and other reaction conditions may be readily selected by one of ordinary skill in the art. Specific procedures are provided in the Synthetic Examples section.

Preferred is a process for the preparation of the macrocyclic compound of formula I from a diene of formula III, wherein a catalyst of formula IV is employed, in which

L is a trihydrocarbylphosphine group, preferably a tri-(C₁₋₆ alkyl)-phosphine or a tri-(C₃₋₈ cycloalkyl)-phospine group, in particular a tricyclohexylphosphine group; or a group of formula

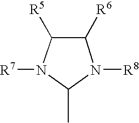

wherein

R⁵ and R⁶ each independently represent a hydrogen atom or a C₁₋₆ alkyl, C₂₋₆ alkenyl, C₆₋₁₂ aryl or C₆₋₁₂ aryl-C₁₋₆ alkyl group, preferably a hydrogen atom; or

R⁵ and R⁶ together form a double bond; and

R⁷ and R⁸ each independently represent a hydrogen atom or a C₁₋₆ alkyl, C₂₋₆ alkenyl, C₆₋₁₂ aryl or C₆₋₁₂ aryl-C₁₋₆ alkyl group, preferably a phenyl group which may be substituted by one, two or three groups selected from halogen atom, C₁₋₆ alkyl and C₁₋₆ alkoxy groups;

X¹ and X² each independently represent a halogen atom, preferably a chlorine atom; and

R⁴ represents a C₁₋₆ alkyl group, preferably a branched C₃₋₆ alkyl group.

More preferred are ruthenium catalysts of formula IV, wherein the nitro group is attached in the para-position with respect to the point of attachment of the alkoxy group R⁴—O—.

Particularly preferred is a process for the preparation of a macrocyclic compound of formula I, wherein the ruthenium catalyst is a compound of formula IVA

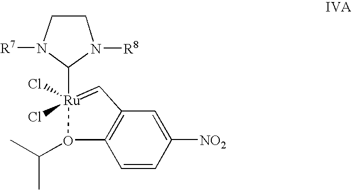

wherein R⁷ and R⁸ represent a trimethylphenyl group, in particular mesityl group.

Furthermore preferred is a process for the preparation of a macrocyclic compound of formula I according to the present invention, wherein the metathesis reaction is carried out in the presence of a diluent in a temperature range from 40 to 120° C., preferably from 60 to 100° C., in particular at about 80° C.

In another preferred embodiment of the present invention the methathesis reaction is carried out in the presence of a diluent selected from the group consisting of alkanes, such as n-pentane, n-hexane or n-heptane, aromatic hydrocarbons, such as benzene, toluene or xylene, and chlorinated hydrocarbons such as dichloromethane, trichloromethane, tetrachloromethane or dichloroethane.

Furthermore preferred is a process for the preparation of a macrocyclic compound of formula I, wherein the molar ratio of the diene compound of formula III to the catalyst of formula IV ranges from 1000:1 to 100:1, preferably from 500:1 to 110:1, in particular from 1:250 to 1:150.

As a rule the process for the preparation of a macrocyclic compound of formula I is carried out at a ratio of the diene compound of formula III to diluent in the range from 1:400 by weight to 1:25 by weight, preferably from 1:200 by weight to 1:50 by weight, in particular from 1:150 by weight to 1:75 by weight.

Furthermore preferred is a process for the preparation of a macrocyclic compound of formula I, wherein R₁ moiety is a group of formula (i)

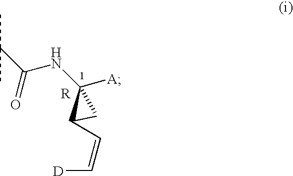

R² is a group of formula II, and

W is N;

R²¹ is H, C₁₋₆ alkyl, C₁₋₆ alkoxy, hydroxy, chloro;

R²² is H, C₁₋₆ thioalkyl, C₁₋₆ alkoxy, phenyl or Het selected from the group consisting of:

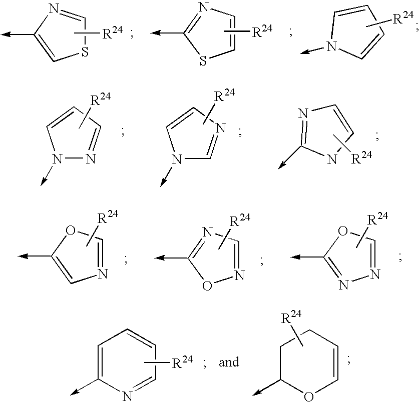

wherein R²⁴ is H, C₁₋₆ alkyl, NH—R²⁵, NH—C(O)—R²⁵; NH—C(O)—NH—R²⁵, wherein each R²⁵ is independently: H, C₁₋₆ alkyl, or C₃₋₆ cycloalkyl;

or NH—C(O)—OR²⁶, wherein R²⁶ is C₁₋₆ alkyl;

R²⁸ is H, bromine or methyl, preferably H or

R² is a leaving group of formula —OSO₂—R²⁷,

wherein R²⁷ is selected from p-toluyl, p-bromophenyl, p-nitrophenyl, methyl, trifluoromethyl, perfluorobutyl and 2,2,2-trifluoroethyl.

In another specific embodiment of the compounds of formula (I), wherein R₁ moiety is a group of formula (i);

A is a carboxylic acid or a pharmaceutically acceptable salt or ester thereof, most preferably COOH;

W is N;

R²¹ is C₁₋₃ alkoxy;

R²² is

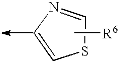

wherein R⁶ is NH—(CO)ₘ—(C₁₋₄alkyl) or NH—(CO)ₘ—(C₃₋₆cycloalkyl), with m being 0 or 1, preferably 0;

R²⁸ is H or methyl, preferably H;

R³ is NH—C(O)—OR¹⁰, wherein R¹⁰ is butyl, cyclobutyl or cyclopentyl;

D is a 5-atom saturated alkylene chain; and

A is a carboxylic acid or a pharmaceutically acceptable salt or ester thereof.

The following tables list compounds representative of the compounds of formula (I). A compound of the formula below:

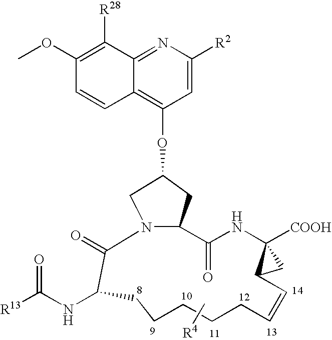

wherein the bond from position 14 to the cyclopropyl group is syn to the COOH, said 13, 14 double bond is cis, R²⁸ is H and R¹³, R⁴ and R² are defined as follows:

TABLE 1

| Cpd # | R¹³: | R⁴: | R²: |
|---|---|---|---|
| 801 | 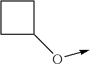 | H | 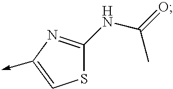 |
| 804 | 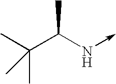 | H | 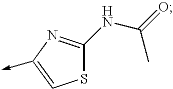 |
| 805 | 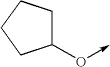 | H | 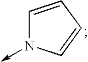 |
| 807 | 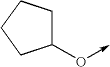 | H | OEt; |
| 808 | 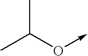 | H | OEt; |
| 809 | 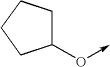 | H | 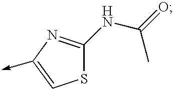 |
| 810 | 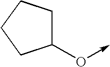 | H | 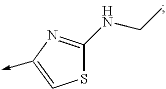 |
| 811 | 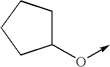 | H | 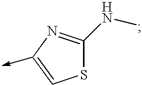 |
| 812 | 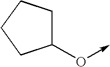 | H | 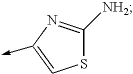 |
| 814 | 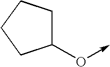 | H | 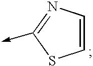 |
| 815 | 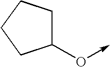 | H | 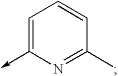 |
| 816 | 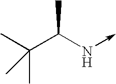 | H | 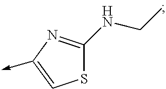 |
| 817 | 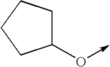 | H | 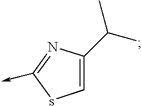 |
| 818 | 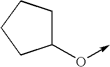 | H | 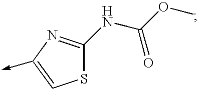 |
| 819 | 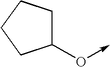 | H | 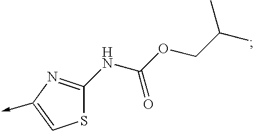 |
| 820 | 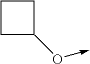 | H | 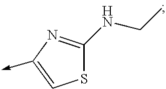 |
| 821 | 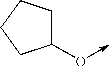 | H | 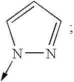 |
| 822 | 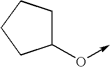 | H | 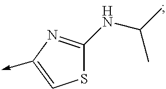 |
| 823 | 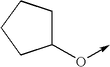 | H | 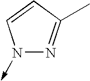 |
| 824 | 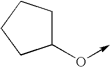 | 10-(R)Me | OEt; |
| 825 | 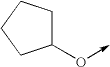 | H | 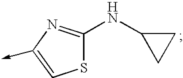 |
| 826 | 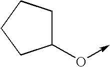 | H | 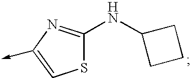 |
| 827 | 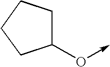 | H | 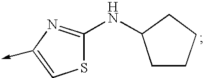 |
| and 828 | 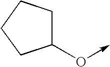 | H | 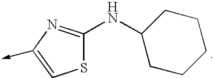 |

or R²⁸ is Methyl and the bond from position 14 to the cyclopropyl group is syn to the COOH, said 13, 14 double bond is cis, and R¹³, R⁴ and R² are defined as follows

TABLE 2

| | | | |
|---|---|---|---|
| Cpd # | R¹³: | R⁴: | R²: |

| | | | |
|---|---|---|---|
| 801′ | 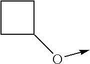 | H | 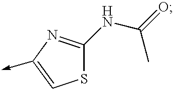 |
| 804′ | 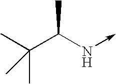 | H | 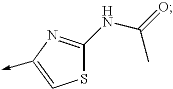 |
| 805′ | 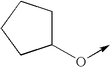 | H | 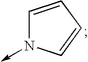 |
| 807′ | 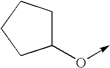 | H | OEt; |
| 808′ | 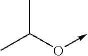 | H | OEt; |
| 809′ | 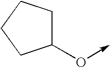 | H | 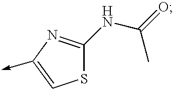 |
| 810′ | 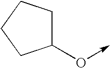 | H | 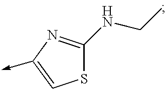 |
| 811′ | 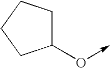 | H | 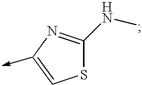 |
| 812′ | 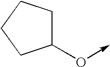 | H | 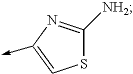 |
| 814′ | 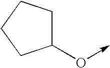 | H | 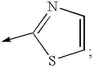 |
| 815′ | 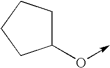 | H | 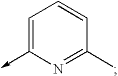 |
| 816′ | 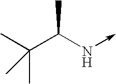 | H | 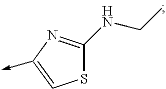 |
| 817′ | 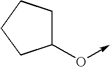 | H | 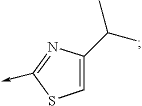 |
| 818′ | 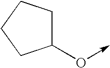 | H | 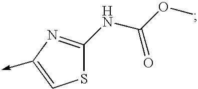 |
| 819′ | 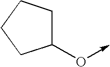 | H | 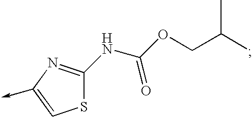 |
| 820′ | 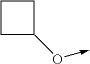 | H | 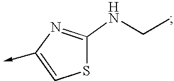 |
| 821′ | 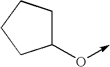 | H | 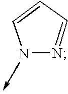 |
| 822′ | 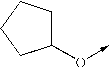 | H | 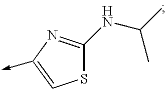 |
| 823′ | 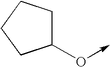 | H | 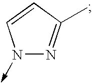 |
| 824′ | 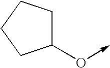 | 10- (R) Me | OEt; |
| 825′ | 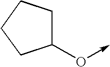 | H | 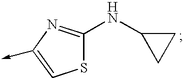 |
| 826′ | 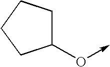 | H | 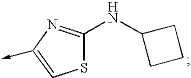 |
| 827′ | 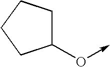 | H | 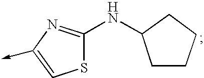 |
| 828′ | 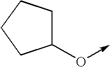 | H | 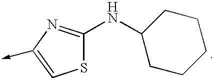 |
| 829′ | 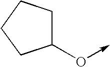 | H | 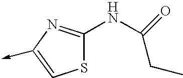 |
| and 830′ | 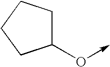 | H | 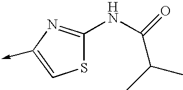 |

A specific representative compound from the table 1 is Compound No. 822.

Additional specific compounds that are representative of the compounds of formula (I) may be found in WO 00/59929 and U.S. Pat. No. 6,608,027, both of which are herein incorporated by reference.

Another aspect of the present invention is a process for the preparation of a macrocyclic compound of formula IA

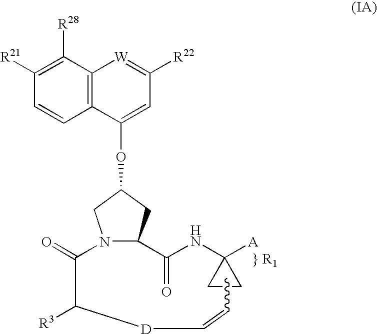

wherein R₁, R³, R²¹, R²², R²⁸, W, A and D have the meaning given for formula I, which comprises the following steps:

(i) macrocycling of a diene compound of formula III

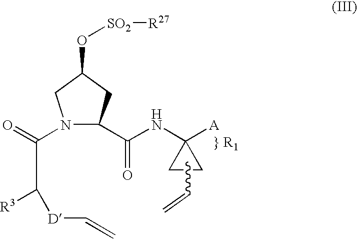

wherein R₁, R³, R²⁷ and A are as defined hereinbefore; and D′ represents a 3 to 7-atom saturated alkylene chain; in the presence of the ruthenium catalyst of formula IV as defined above; and

(ii) reacting the resulting macrocyclic compound of formula I,

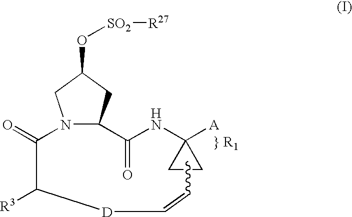

wherein A, R₁, R³, R²⁷ and D are as defined hereinbefore; with a compound of formula V,

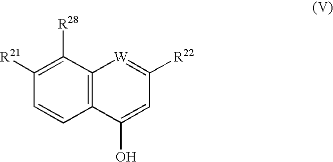

wherein R²¹, R²², R²⁸ and W are as defined hereinbefore.

The hydroxyl-substituted quinoline compounds of formula (V) are known, e.g., from WO 00/59929, WO 00/09543 and WO 00/09558, U.S. Pat. No. 6,323,180 B1 and U.S. Pat. No. 6,608,027 B1.

The catalysts of formula IV can be prepared according to the method described by K. Grela et al., Angew. Chem. Int. Ed. 2002, 41, No. 21 pp. 4038-4040, the complete disclosure of which being incorporated herein by reference. The catalysts of formula IV are preferably prepared by reacting a 2-alkoxy-nitro-stilbene compound of formula V with a ruthenium compound of formula VI in the presence of transition metal salts such as Cu (I) salts in particular CuCl according to the following reaction scheme:

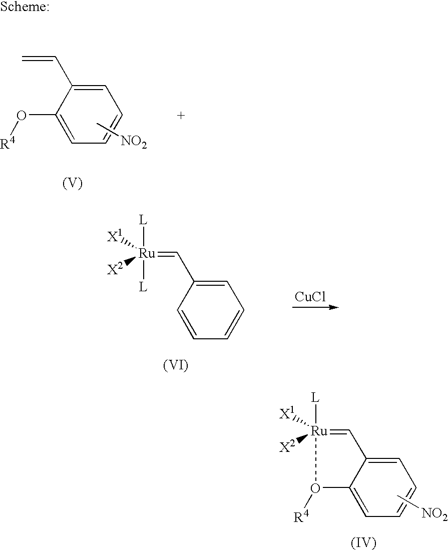

Preferred ruthenium compounds of formula VI for the preparation of the catalysts of formula IV are Grubb's catalyst (L=tricyclohxylphosphine), Nolan's catalyst (L=1,3-dimesityl-dihydro-imidazolin-2-yl) and Scholl's catalyst (L=1,3-dimesitylimidazolidine-2-yl), which can be prepared as described in the International patent application WO 00/71554:

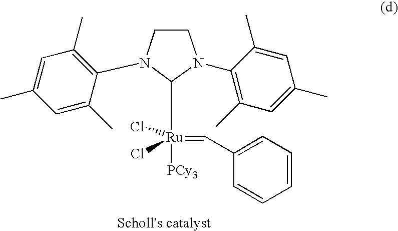

In order that this invention be more fully understood, the following examples of are set forth. These examples are for the purpose of illustrating embodiments of this invention, and are not to be construed as limiting the scope of the invention in any way.

### EXAMPLE 1

### Step A: Preparation of (L)-N-boc-trans-hydroxyprolinol

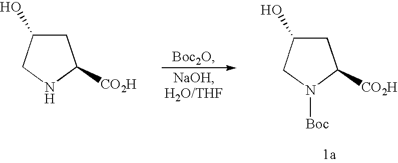

(L)-trans-hydroxyprolinol (249.8 g, 1.905 mol) is dissolved in water (375 ml) and 45% sodium hydroxide solution (203 g, 2.286 mol). tert.-Butanol (106 g) is added. The reaction mixture is heated to 50° C. and the anhydride Boc2O (424 g, 1.943 mol) dissolved in THF (425 ml) is slowly added. After the addition the reaction mixture is kept ½-1 h at 50° C., the THF is distilled off the solution. The pH is adjusted at ca. 3 with conc. HCl (204 g, 2.076 mol) and the product is then extracted with methyl-isobutylketon (MIBK) (1 l) and again with MIBK (375 ml). The organic layer is heated and some of the solvent is distilled off to remove traces of water. The product is crystallized from this solution by adding methylcyclohexane (1.25 l), isolated, washed twice with MCH (375 ml) and dried overnight at 40° C. to yield: 77-78% of 1a as colorless crystals, Fp=126-128° C.

### Step B: Lactonisation

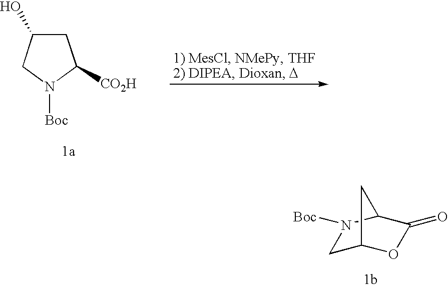

1a (416,3 g, 1.8 mol) is dissolved in THF (2.08 l) and cooled with ice to −5-−10° C. Mesylchloride (392 g, 3.4 mol) and N-methylpyrrolidine (429 g, 5 mol) is added and the mixture stirred for 1½ h at −5° C. The mixture is washed with water and heated up to reflux. Dioxane (2,08 l) is poured in and the THF is distilled off. After cooling down to room temperature, diisopropylethylamine (233 g, 1.8 mol) is added and the mixture is heated to reflux. After 1 h part of the solvent (830 ml) is distilled off, cooled to ambient temperature and a KHSO4-solution (14.4 g in 2.08 l water) is poured in and the solution is allowed to coll down to room temperature. The resulting crystals are isolated with a suction funnel, washed with water and dried overnight at 45° C. to yield 78-82% of 1b as colorless needles, Fp=111° C.

### Step C: Deprotection

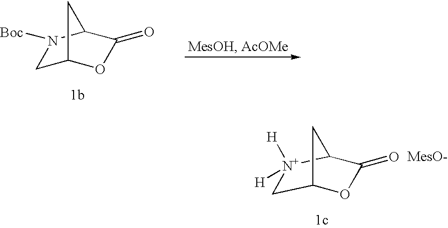

1b (267 g, 1.25 mol) is dissolved in MIBK (1467 ml). The suspension is heated up to 50° C. until 1b is completely dissolved and a part of the solvent (130 ml) is distilled off to remove traces of water. Methansulfonic acid (240 g, 2.5 mol) is added slowly to the reaction mixture. The reaction mixture is allowed to cool to room temperature and the resulting crystals are isolated with a suction funnel, washed twice with acetone (each 400 ml) and dried overnight at 40° C. to yield 93-98% of 1c as colorless crystals, 208-210° C.

### Step D: Synthesis of the Dipeptide

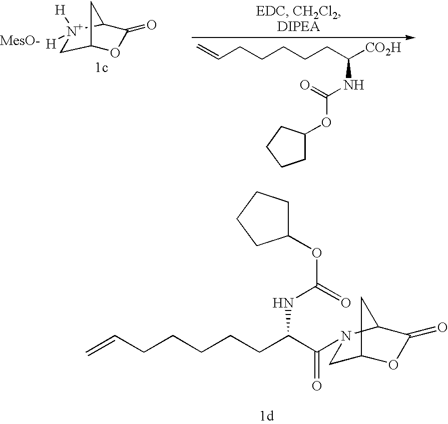

2-(N-Cyclopentyloxycarbonyl-amino)-non-8-enoic acid*DCHA (61.4 g, 132 mmol) is dissolved in toluene (160 ml) and the resulting solution is washed with diluted sulfuric acid (5.3 g in 80 ml water) and water (80 ml). After phase separation, the solution is treated with charcoal and filtered and the resulting solution stored at room temperature. 1c (24.9 g, 119 mmmol) and EDC*HCl (26.8 g, 140 mmol) are suspended in dichloromethane (140 ml) and cooled to room temperature. The suspension is treated with the solution of 2-(N-cyclopentyloxycarbonyl-amino)-non-8-enoic acid generated before. To this suspension, Di-isopropylethylamine (16.3 g, 130 mmol) is slowly added while the reaction is kept under nitrogen at temperatures below 20° C. The suspension is filtered, and the resulting solution is washed water (80 ml), diluted acetic acid (1.3 g in 80 ml water), 5% sodium bicarbonate solution (80 ml) and again with water (80 ml). After phase separation, dichloromethane is distilled off under reduced pressure. The resulting solution can directly be used for the next step. Otherwise, the product can be isolated by crystallization with MCH to yield 95% (GC) of 1d as yellowish solution, Fₚ=58-60° C.

### EXAMPLE 2

### Step A: Preparation of the Tripeptide 2a

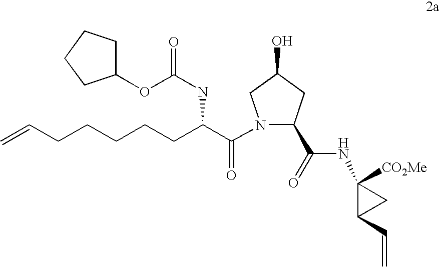

A mixture of methyl 1-amino-2-vinyl-cycloprop-1-ylcarboxylate (10.0 g, 23.7 mmol, 1.0 eq.), 1d (7.6 g, 24.2 mmol, 1.02 eq.) and sodium 2-ethylhexanoate (5.9 g, 35.6 mmol, 1.5 eq.) in water (43 ml) and toluene (12 ml) is stirred at 80° C. for 2 h. For work-up toluene (75 ml) is added at 80° C. After stirring and separation of the aqueous layer, the organic layer is washed with 1M Na2CO3 (3×30 ml), 0.5M HCl (30 ml) and water (2×30 ml). The solvent is removed completely in vacuo to yield: 11.7 g, 22.5 mmol, (95%) of 2a; purity: >95% (peak-area HPLC) as a slightly yellow oil.

### Step B: Brosylation of 2a

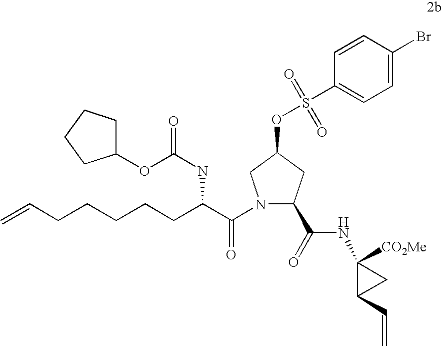

To a mixture of 2a (10.7 g, 18.5 mmol, 1.0 eq.) and DABCO (3.3 g, 29.7 mmol, 1.6 eq.) and toluene (23 ml) a solution of brosyl chloride (6.6 g, 26.0 mmol, 1.4 eq.) in toluene (15 ml) is added slowly at room temperature. The mixture is stirred for 2 h. For work-up the organic layer is washed with 1M Na2CO3 (2×21 ml), diluted with THF (21 ml) and washed with 0.5M HCl (21 ml) and water (2×21 ml). The solvent is removed completely in vacuo to yield 12.3 g, 16.7 mmol of 2b (90%); purity: >95% (peak-area HPLC) as a slightly orange oil. A charcoal treatment of the crude product is possible.

### EXAMPLE 3

### Metathesis of 2b

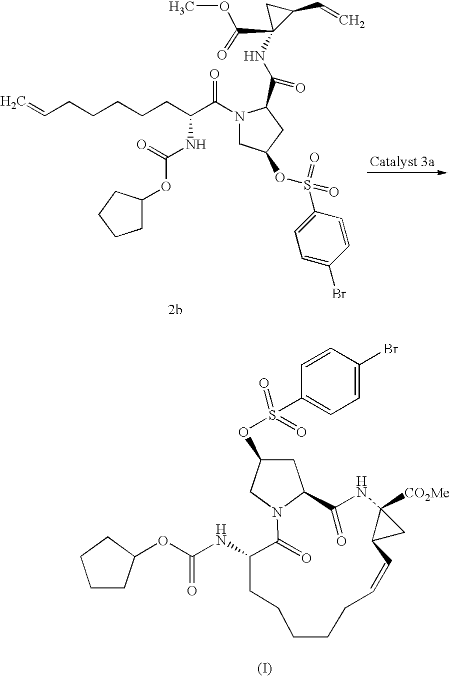

### Step A Preparation of the Catalyst

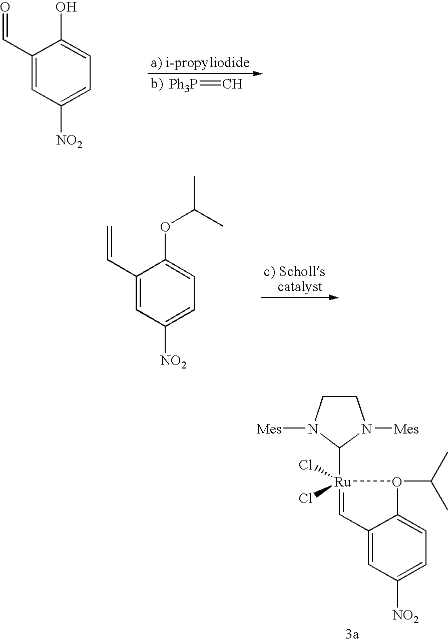

3a Ruthenium Catalyst

The ruthenium catalyst is prepared in accordance with the method disclosed by K. Grela et al., Angew. Chem. Int. Ed. 2002, 41, No. 21 pp. 4038-4040 as follows: 0.8 ml (8 mmol) 2-iodopropane is added to a stirred mixture 1.1 g (8 mmol) of dry powdered potassium carbonate 521 mg of cesium carbonate, 668 mg (4 mmol) 2-hydroxy-5-nitrobenzaldehyde and 25 mL dimethylformaide (DMF). After stirring at ambient is temperature for 24 hours DMF is removed in vacuo and residue is poured into 50 ml of water and extracted four times with 25 ml of tert-butylmethylether (TBME). The combined organic extracts are washed with brine, dried and concentrated in vacuo. The crude product is purified by silica gel column chromatography (cyclohexane:ethyl acetate:8:2) to yield 850 mg of 2-isopropoxy-5-nitrobenzaldehyde as low melting yellow crystals. A solution of n-butyllithium in hexane (1.8 mL, 2.7 mmol, 1.5M) is added to a stirred solution of 932 mg (2.53 mmol) of triphenylmethylphosphonium bromide in 2 mL of tetrahydrofuran (THF) at −78° C. A solution of 379 mg (1.81 mmol) 2-isopropoxy-5-nitrobenzaldehyde in 2 mL THF is added thereto at −78° C. The reaction mixture is allowed to warm up to ambient tmperature and stirred at ambient temperature for 10 hours.

The reaction mixture is poured into a saturated solution of ammonium chloride and diluted with 100 ml of TBME. The solid material is filtered off and the crude product is passed through a short column of silica, concentrated and purified on silica-gel using column chromatography(cyclohexane:ethyl acetate:8:2) to yield 236 mg (63%) of 2-isopropoxy-5-nitrostilbene as a pale yellow oil.

A solution of 38 mg (0.18 mmol) of 2-isopropoxy-5-nitrostilbene in 4 mL of dichloromethane is added to a mixture of 153 mg (0.18 mmol) of Scholl's catalyst, 18 mg (0.18 mmol) CuCl and 18 mL dichloromethane and stirred under inert gas atmosphere at 30° C. for 1 hour. The resulting reaction mixture is concentrated in vacuoand piurified by column chromatography on silica. Elution with cyclohexane:ethyl acetate (5:2) yields 100 mg (83%) of the catalyst 3a as a green microcrystalline solid.

The spectroscopic data are in good agreement with those disclosed by K. Grela et al., loc. cit.

### Step B Preparation of THP Solution

23.5 g Tetrakishydroxymethylphosphoniumchlorid (80%, 98.7 mmol) is dissolved in isopropanol (35 ml) under a nitrogen atmosphere. Then 12.1 g (98.7 mmol) of a 45% KOH solution is added within 5 min while the solution is cooled (temperature 20-25° C.). After stirring the suspension for another 30 min under nitrogen, the mixture is filtered and the inorganic residue is washed with 20 ml of degassed isopropanol. The combined isopropanol solution is stored under a nitrogen atmosphere until use.

### Step C Metathesis Reaction

810 ml of toluene are degassed by bubbling through nitrogen. 7.02 g (9.5 mmol) of 2b are dissolved in 10 ml of degassed toluene and added into the reaction flask. The solution is heated up to 80° C. and 0.032 g (0.048 mmol) of the freshly prepared catalyst 3a is added under nitrogen in four portions over a period of 3 hours. After stirring for further 60 min at the same temperature the conversion is checked by HPLC. After cooling to 60° C. 2.3 g (2.8 mmol) of the THP suspension 3b is added to the reaction mixture. After stirring for 5 h at 60° C. the mixture is cooled to room temperature and extracted twice with 40 ml of degassed water, 40 ml of 0.5 M HCl, 40 ml of 0.5 M NaHCO₃ solution, and 40 ml of water. Approx. 695 ml of toluene are distilled of at 50° C. in vacuo (150 mbar) and the residue is treated at 50° C. with 1.4 g of charcoal (Acticarbon L2S). The remaining liquid is added to 210 ml of pre-cooled methylcyclohexane (5° C.). After stirring for further 60 min at 5° C. the precipitate is filtered and washed with 100 ml of methylcyclohexane (twice).

The white solid is dried in vacuo at 30° C. to yield 5.78 g (85.6%) of (I) as an almost white powder. EXAMPLE 4

### Synthesis of Compound 4

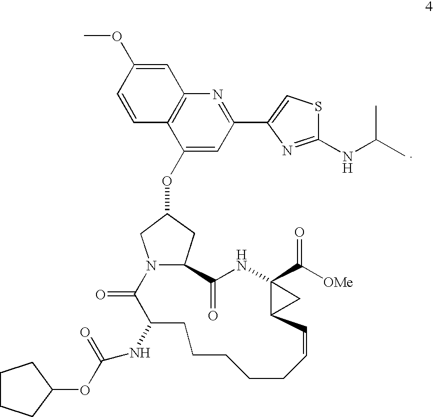

A mixture of (1 eq.) Cs2CO3, (1 eq.) 2-(2-isopropylaminothiazol-4-yl)-4-hydroxy-7-methoxyquinoline and I (1 eq.) in N-methylpyrrolidone (NMP) is stirred for 8 h at 55 to 65° C. After completion of the reaction the mixture is diluted with ethyl acetate and extracted with 2,5% NaHCO3 solution. The organic layer is extracted three times with a mixture of a 2,5% solution of NaHCO3 and NMP. The organic layer is treated with charcoal, filtered, and the product is crystallized by the addition of n-heptane (or methylcyclohexane). The suspension is cooled to 5° C., the precipitate is filtered and washed with ethyl acetate/n-heptane (or ethyl acetate/methylcyclohexane) and dried in vacuo to yield: 60-70% of 4 as white crystalls. If necessary (quality) the product can be re-crystallized from ethyl acetate/methylcyclohexane. EXAMPLE 5

### Saponification of 4/Preparation of 822

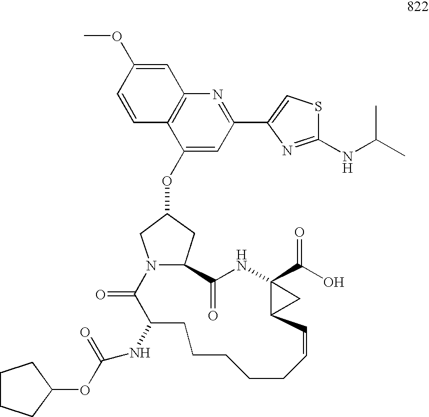

20 g (0.025 mol) of 4 is dissolved in 160 ml of THF and 2.45 g (0.0583 mmol) of LiOH*H2O is added to the solution. After the addition of 54 ml of water the reaction mixture is stirred for at least 8 h at a temperature of 40-45° C. After complete conversion (HPLC) the mixture is cooled to 20-25° C. After separation of the layers (a small aqueous phase is separated off) 54 ml of ethanol is added to the organic layer and the pH is adjusted to pH 5.5-5.7 by the addition of 1M HCl solution. The mixture is warmed to 40-45° C. and 80 ml of water are added over a period of at least 30 min (40-45° C.). The mixture is stirred for further 60 min at a temperature of 40-45° C. Further 80 ml of water are added at 40-45° C. over a period of at least 30 min and the mixture is stirred for another 60 min at the same temperature. The suspension is cooled to 20-25° C. and stirred at this temperature for 1 h. After filtration the precipitate is washed three times by 20 ml of water and dried in vacuo at 35° C. (slight stream of N2) to yield 17.7-18.7 g of crude 822 (90-95%).

10 g (0.0129 mol) crude 822 are dissolved in 100 ml of ethanol at 20-25° C. Then the solution is treated with charcoal (5-20%), filtered and added to 240 ml of water at 70-75° C. over a period of 1 h. The mixture is cooled to 25-30° C. over a period of at least 1 h. After filtration the precipitate is washed with 40 ml of a 1.7/1 mixture of ethanol/water and dried in vacuo at 45° C. (slight stream of nitrogen) to yield: 9.2-9.7 g of pure 822 (92-97%), which contains between 3 and 5% of water.

1. A process for the preparation of a macrocyclic compound of formula I 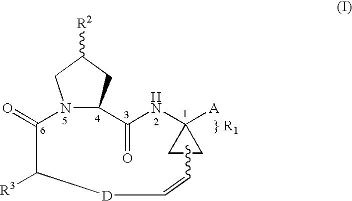
wherein
R² is a hydroxy group, a leaving group or a group of formula II 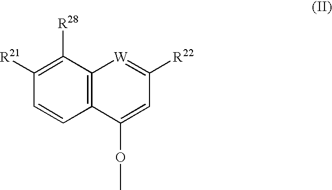
W is CH or N,
R²¹ is H, halo, C₁₋₆ alkyl, C₃₋₆ cycloalkyl, C₁₋₆ haloalkyl, C₁₋₆ alkoxy, C₃₋₆ cycloalkoxy, hydroxy, or N(R²³)₂,
wherein each R²³ is independently H, C₁₋₆ alkyl or C₃₋₆ cycloalkyl;
R²² is H, halo, C₁₋₆ alkyl, C₃₋₆ cycloalkyl, C₁₋₆ haloalkyl, C₁₋₆ thioalkyl, C₁₋₆ alkoxy, C₃₋₆ cycloalkoxy, C₂₋₇ alkoxyalkyl, C₃₋₆ cycloalkyl, C_{6 or 10} aryl or Het, wherein Het is a five-, six-, or seven-membered saturated or unsaturated heterocycle containing from one to four heteroatoms selected from nitrogen, oxygen and sulfur;
said cycloalkyl, aryl or Het being substituted with R²⁴, wherein
R²⁴ is H, halo, C₁₋₆ alkyl, C₃₋₆ cycloalkyl, C₁₋₆ alkoxy, C₃₋₆ cycloalkoxy, NO₂, N(R²⁵)₂, NH—C(O)—R²⁵; or NH—C(O)—NH—R²⁵, wherein each R²⁵ is independently: H, C₁₋₆ alkyl or C₃₋₆ cycloalkyl; or
R²⁴ is NH—C(O)—OR²⁶ wherein R²⁶ is C₁₋₆ alkyl or C₃₋₆ cycloalkyl;
R²⁸ is H, halo or C₁₋₆ alkyl,
R³ is hydroxy, NH₂, or a group of formula —NH—R³¹, wherein R³¹ is C_{6 or 10} aryl, heteroaryl, —C(O)—R³², —C(O)—NHR³² or —C(O)—OR³²,
wherein R³² is C₁₋₆ alkyl or C₃₋₆ cycloalkyl;
D is a 3 to 7-atom saturated alkylene chain; and
A is an amide of formula —C(O)—NH—R⁵, wherein R⁵ is selected from the group consisting of: C₁₋₈ alkyl, C₃₋₆ cycloalkyl, C_{6 or 10} aryl, C₇₋₁₆ aralkyl; and
SO₂R^{5A} wherein R^{5A} is C₁₋₈ alkyl, C₃₋₇ cycloalkyl or {C₁₋₆ alkyl-C₃₋₇ cycloalkyl}, or
A is a carboxylic acid or a pharmaceutically acceptable salt or ester thereof;
which process comprises subjecting a diene compound of formula III 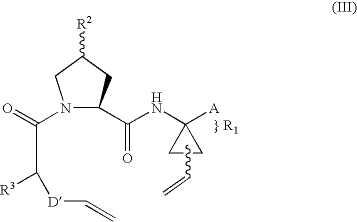
wherein R², R³ and A are as defined hereinbefore; and
D′ represents a 3 to 7-atom saturated alkylene chain;
to a metathesis cyclization reaction in the presence of a ruthenium catalyst of formula IV: 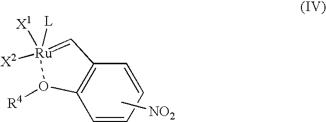
wherein
X¹ and X² each independently represent an anionic ligand;
L represents a neutral electron donor ligand; and
R⁴ represents a C₁₋₆ alkyl, C₂₋₆ alkenyl or C₆₋₁₂ aryl-C₁₋₆ alkyl group.

2. A process according to claim 1 for the preparation of a macrocyclic compound of formula I, wherein L of formula IV is a trihydrocarbylphosphine group or a group of formula 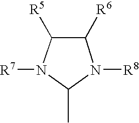
wherein
R⁵ and R⁶ each independently represent a hydrogen atom or a C₁₋₆ alkyl, C₂₋₆ alkenyl, C₆₋₁₂ aryl or C₆₋₁₂ aryl-C₁₋₆ alkyl group; or
R⁵ and R⁶ together form a double bond; and
R⁷ and R⁸ each independently represent a hydrogen atom or a C₁₋₆ alkyl, C₂₋₆ alkenyl, C₆₋₁₂ aryl or C₆₋₁₂ aryl-C₁₋₆ alkyl group, each optionally substituted by one, two or three groups independently selected from halogen, C₁₋₆alkyl and C₁₋₆alkoxy;
X¹ and X² each independently represent a halogen atom; and
R⁴ represents a C₁₋₆ alkyl group.

3. A process according to claim 1 for the preparation of a macrocyclic compound of formula I, wherein the ruthenium catalyst is a compound of formula IVA 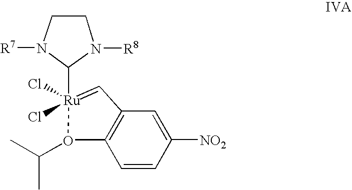
wherein R⁷ and R⁸ represent a mesityl group.

4. A process according to claim 1 for the preparation of a macrocyclic compound of formula I, wherein R₁ moiety is a group of formula (i) 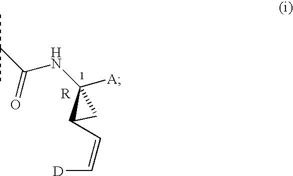
R² is a group of formula II,; and
W is N;
R²¹ is H, C₁₋₆ alkyl, C₁₋₆ alkoxy, hydroxy, chloro;
R²² is H, C₁₋₆ thioalkyl, C₁₋₆ alkoxy, phenyl or Het selected from the group consisting of: 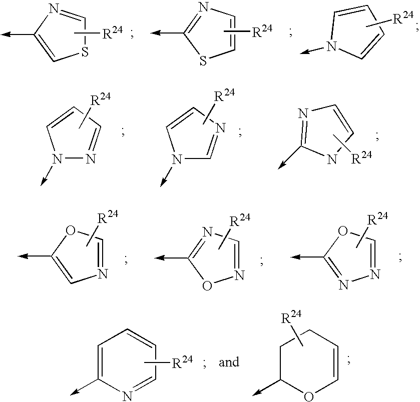
wherein R²⁴ is H, C₁₋₆ alkyl, NH—R²⁵, NH—C(O)—R²⁵; NH—C(O)—NH—R²⁵, wherein each R²⁵ is independently: H, C₁₋₆ alkyl, or C₃₋₆ cycloalkyl;
or NH—C(O)—OR²⁶, wherein R is C₁₋₆ alkyl;
R²⁸ is H, bromine or methyl; or
R² is a leaving group of formula —OSO₂—R²⁷, wherein R²⁷ is selected from p-toluyl, p-bromophenyl, p-nitrophenyl, methyl, trifluoromethyl, perfluorobutyl and 2,2,2-trifluoroethyl.

5. A process according to claim 1 for the preparation of a macrocyclic compound of formula I, wherein metathesis reaction is carried out in the presence of a diluent in a temperature range from 40 to 120° C.

6. A process according to claim 1 for the preparation of a macrocyclic compound of formula I, wherein metathesis reaction is carried out in the presence of a diluent selected from alkanes, aromatic hydrocarbons, and chlorinated hydrocarbons.

7. A process according to claim 1 for the preparation of a macrocyclic compound of formula I, wherein the molar ratio of the diene compound of formula III to catalyst of formula IV ranges from 1000:1 to 100:1.

8. A process according to claim 1 for the preparation of a macrocyclic compound of formula I, wherein the ratio of the diene compound of formula III to diluent ranges from 1:400 by weight to 1:25 by weight.

9. A process for the preparation of a macrocyclic compound of formula I 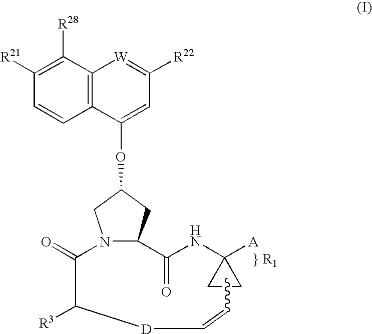
wherein R³, R²¹, R²², R²⁸, W, A and D are as defined in claim 1, which comprises the following steps:
(i) cyclizing a diene compound of formula III 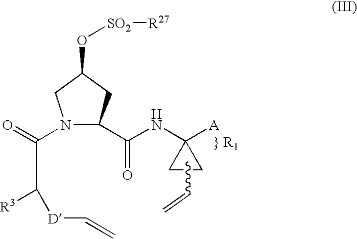
wherein R³ and A are as defined in claim 1, and R²⁷ is selected from p-toluyl, p-bromophenyl, p-nitrophenyl, methyl, trifluoromethyl, perfluorobutyl and 2,2,2-trifluoroethyl; and
D′ represents a 3 to 7-atom saturated alkylene chain;
in the presence of the ruthenium catalyst of formula IV: 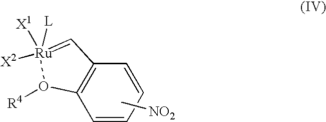
wherein
X¹ and X² each independently represent an anionic ligand;
L represents a neutral electron donor ligand; and
R⁴ represents a C₁₋₆ alkyl, C₂₋₆ alkenyl or C₆₋₁₂ aryl-C₁₋₆ alkyl group; and
(ii) reacting the resulting macrocyclic compound of formula I, 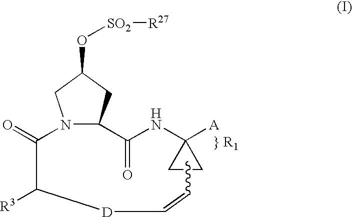
wherein A, R³ and D are as defined in claim 1 , and R²⁷ is as defined above in step (i), with a compound of formula V, 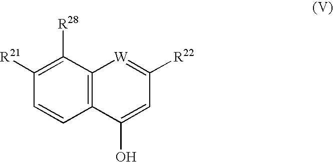
wherein R²¹, R²², R²⁸ and W are as defined in claim 1.
